(19) United States
(12) Patent Application Publication
(10) Pub. No.: US 2026/0191449 A1
(43) Pub. Date: Jul. 9, 2026

(54) ELECTRIC IMPEDANCE MYOGRAPHY FOR CHARACTERIZING AND TRACKING BLADDER AND PELVIC FLOOR DISORDERS

(71) Applicants: The Children's Medical Center Corporation, Boston, MA (US); Beth Israel Deaconess Medical Center, Inc., Boston, MA (US)

(72) Inventors: Hsin-Hsiao Scott Wang, Chestnut Hill, MA (US); Seward B. Rutkove, Brookline, MA (US)

(73) Assignees: The Children's Medical Center Corporation, Boston, MA (US); Beth Israel Deaconess Medical Center, Inc., Boston, MA (US)

(21) Appl. No.: 19/130,505

(22) PCT Filed: Nov. 17, 2023
(86) PCT No.: PCT/US2023/080214

§ 371 Date: May 15, 2025

Related U.S. Application Data
(60) Provisional application No. 63/426,250, filed on Nov. 17, 2022.

(51) Int. Cl. A61B 5/20; A61B 5/00; A61B 5/0538; A61B 5/296; A61B 5/391

(52) U.S. Cl. CPC A61B 5/202; A61B 5/0538; A61B 5/296; A61B 5/391; A61B 5/4839; A61B 5/685; A61B 5/6852

(57) ABSTRACT

Devices and methods for evaluating and treating neurogenic bladder (NB) are disclosed. In some embodiments, a probe may include microneedles for gently puncturing the bladder detrusor tissue of a test subject. The microneedles may non-invasively collect a series of electric impedance myography (EIM) measurements to evaluate the condition of the bladder and assist in diagnosis. In some embodiments, the probe may provide information regarding the tissue's structural and biophysiological condition, in a real-time, quantitative, and objective manner. In some embodiments, the probe may assist in identifying optimal treatment (e.g., Botox or biomaterials injection) sites for NB by providing real-time results.

### FEDERALLY SPONSORED RESEARCH

This invention was made with government support under DK060442-16 awarded by the National Institutes of Health. The government has certain rights in the invention.

### FIELD

Disclosed embodiments are related to electric impedance myography and related systems of use.

### BACKGROUND

Neurogenic bladder (hereinafter referred to as “NB”) is a condition that widely affects people of all ages. It results in urinary incontinence, lower urinary tract symptoms such as urinary frequency, urgency, and pain, urinary retention, and urinary tract infection. NB is fundamentally caused by aberrant or interrupted neural control of the bladder and is commonly seen in patients with a wide range of conditions, including, diabetes, pelvic surgery, and those with primary neurologic conditions such as, multiple sclerosis, spinal cord injury, and spina bifida. Other conditions that may affect the bladder include other non-neurological entities, including bladder stones, benign prostatic hypertrophy, stress incontinence, and other causes of bladder pathologic changes. In the United States alone, NB is associated with a significant healthcare burden with billions of dollars spent annually.

### SUMMARY

In some embodiments, methods of evaluating bladder function include puncturing bladder detrusor tissue of a subject with a plurality of microneedles arranged at a proximal end of a probe, and collecting at least one electric impedance myography measurement from the bladder detrusor tissue.

In some embodiments, methods of evaluating bladder function include puncturing bladder detrusor tissue of a subject with a plurality of microneedles arranged at a proximal end of a probe, collecting at least one electric impedance myography measurement from the bladder detrusor tissue, and injecting a material into the bladder detrusor tissue.

In some embodiments, an apparatus for evaluating bladder function includes a probe comprising a plurality of microneedles configured to puncture bladder detrusor tissue of a subject, wherein the probe is configured to collect at least one electric impedance myography measurement from the bladder detrusor tissue.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1B show, according to some embodiments, a system for electric impedance myography (EIM) of a test subject;

FIGS. 2A-2B show an EIM probe according to some embodiments;

FIGS. 3A-3B show an EIM probe according to other embodiments;

FIGS. 4A-4B show an EIM probe according to other embodiments;

FIGS. 5A-5B show an EIM probe according to other embodiments still;

FIGS. 6A-6B show exemplary impedance phase measurements using an EIM probe, according to some embodiments;

FIGS. 7A-7B show exemplary impedance resistance measurements using an EIM probe, according to some embodiments;

FIGS. 8A-8B show exemplary impedance reactance measurements using an EIM probe, according to some embodiments;

FIG. 9 shows exemplary impedance phase measurements using an EIM probe, according to some embodiments;

FIG. 10 shows exemplary physiological measurements of a group of test subjects;

FIG. 11 shows an exemplary void spotting assay of a group of test subjects;

FIGS. 12A-12B show exemplary histological slides of a pair of test subjects;

FIG. 13 shows exemplary collagen density measurements of a group of test subjects;

FIG. 14 shows exemplary mRNA expression of a group of test subjects;

FIG. 15 shows exemplary analysis of mRNA expression shown in FIG. 14;

FIG. 16 shows a portion of an EIM probe configured to move between a contracted configuration and an expanded configuration, according to some embodiments;

FIG. 17 shows an EIM probe including a patch, according to some embodiments;

FIG. 18 shows a schematic view of an EIM probe arranged to contact a subject's bladder, according to some embodiments; and

FIG. 19 shows a schematic view of a plurality of impedance electrodes positioned at a perineum of a subject, according to some embodiments.

### DETAILED DESCRIPTION

Conventionally, bladder function in patients with NB is conducted using urodynamic studies, which examine the effectiveness of the urinary system (e.g., bladder, sphincters, and urethra) at controlling urine flow. Urodynamic studies typically involve measurements of bladder nerve and muscle function, pressure, and urine flow rate, among others, through various tests.

The Inventors have recognized that the variety of urodynamic testing methodologies, in combination with arbitrarily defined metrics used for evaluation result in subjective, irreproducible, and generally qualitative results. The inter-observer variability of the urodynamic studies poses further challenges in achieving quantifiable and reproducible results. In addition, many of the urodynamic tests can be highly invasive (e.g., multichannel cystometry, in which pressurized catheters measure the contractile forces in the rectum and the urethra) and time-consuming (e.g., urine culture tests, which provide results in several days). Other diagnostic challenges include subjective patient-reported symptoms, which can further complicate diagnosis. All of these factors, and others, can result in large practice variation, which can result in inadequate clinical care for NB patients. The Inventors have therefore recognized a need for a reliable, objective, repeatable, and non-invasive method of evaluating bladder function.

The Inventors have also recognized that conventional treatment methods for NB, including bladder detrusor injections (e.g., Botox or biomaterials) for poorly compliant neurogenic bladders, may also suffer from inaccuracy, leading to inconsistent clinical outcomes. The bladder is a complex biological organ with distinct histologic layers as well as anisotropic heterogeneity in detrusor smooth muscle contraction. These local biophysiological and structural variations are not necessarily apparent to treating clinicians, who may only have access to gross endoscopic views of the bladder during injection. The current standard of care, in some cases, includes a clinician subjectively picking sites according to gross anatomic landmarks in the bladder and blindly injecting as many sites as possible without causing systemic toxicity, which can be inefficient and reduce the effectiveness of the overall treatment. Therefore, the Inventors have further recognized a need for reliable and objective strategies to help a clinician select optimal injection location.

Based on the foregoing, the Inventors have appreciated the benefits associated with a system for evaluating and/or treating NB in a real-time, quantifiable manner. The system may quantitatively assess various biophysiological and structural changes associated with NB with high reproducibility to enhance clinical care and diagnostics, without relying on clinician-specific metrics or subjective evaluation. The Inventors have also recognized the benefits associated with a system for evaluating and/or treating NB in a non-invasive manner, which may enhance the clinical experience of the patient, and reduce the risks and costs associated with invasive techniques. The system may also assist in providing real-time treatment response monitoring to enhance the accuracy of NB treatments. However, instances in which different benefits are offered by the systems and methods disclosed herein are also possible.

In some embodiments, the systems described herein may employ probes to collect electric impendence myography (EIM) measurements to facilitate diagnosis and treatment of bladder function, including NB. EIM involves the application of a weak high-frequency electrical current to a target tissue and collecting the resultant signals. Without wishing to be bound by theory, the target tissue may be modeled as an RC circuit, the resistance of the tissue depending on the resistance of various fluids within the tissue and the reactance of the tissue depending on the capacitive behavior of the various cell membranes.

EIM is conventionally used as a tool to facilitate neuromuscular diagnosis and monitoring for diseases such as amyotrophic lateral sclerosis (ALS) and Duchenne muscular dystrophy (DMD), by elucidating tissue integrity and atrophy or degradation over time. EIM has been shown to exhibit high sensitivity to structural and biophysical properties in a real-time manner with numerical data, without the need for complex image analysis or detailed operator interpretation. Furthermore, EIM typically requires low operational costs, can collect data non-invasively, and provides highly reproducible results. These benefits render EIM a good candidate for clinical diagnosis and treatment. However, the Inventors have appreciated that the differences between musculoskeletal and bladder tissues may pose challenges in applying EIM data collection methods to bladder tissue.

In some embodiments, the systems described herein may collect and subsequently process EIM measurements from bladder tissue in a real-time manner, providing accurate and reproducible results in comparison to conventional techniques. The probes may include EIM sensors and electrical lead into small caliber needles (e.g., microneedles) that can be passed via existing endoscopic instruments, including, but not limited to, cystoscopes. The microneedles may then be gently punctured into the bladder detrusor muscle tissue in order to provide real-time measurements of various impedance parameters for the target bladder detrusor. In some embodiments, these measurements may be used to evaluate bladder detrusor structural changes, monitor drug effects, facilitate tissue plane selection for intervention, and predict interventional or treatment success due to its non-invasive and real-time nature.

The EIM probes of the present disclosure may measure any suitable impedance variables, including, but not limited to resistance, reactance, and phase. Without wishing to be bound by theory, impedance resistance may include the measurement of difficulty passing current through the tissue, impedance reactance may include the measurement of the capacitive effects of the cell membranes, and impedance phase may include a trigonometric proportion of the resistance and reactance, such that phase may be equivalent to the arctangent of the quotient of the reactance and the resistance.

As will be described in greater detail below, exemplary experiments may verify the validity of EIM measurements to evaluate NB, using female mice with spinal cord injuries. In total, 28 female, X weeks old, C57BL/6J mice may be used, twenty of which may undergo spinal cord transection; and eight of which may be used as healthy controls. Following euthanization, each mouse's bladder may be measured in situ with an EIM probe. The bladder tissue may then be processed for molecular and histologic study. The results of the non-limiting exemplary experiments described below may suggest that EIM signatures (e.g., lower reactance, resistance, and phase values across most frequencies compared to control) for NB model bladders corresponded to conventional NB evaluation techniques, including physiological measurements, collagen density evaluation, and mRNA measurements, among others.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

Without wishing to be bound by theory, in spinal cord injury (SCI), micturition reflex may be eliminated in the acute phase with storage dysfunction (e.g. detrusor overactivity, bladder hypertonicity) and voiding dysfunction (e.g. detrusor sphincter dyssynergia). These changes may induce bladder remodeling in a three phase manner, which may include hypertrophy, compensation, and decompensation. In the initial phase, smooth muscle cell hypertrophy within the detrusor may be a hallmark of the low flow/high pressure system seen in outlet obstruction. In addition, extracellular matrix remodeling contributes to inter- and intra-fascicular collagen and elastic fiber deposition. As noted previously, the Inventors have recognized that the murine SCI model may be an ideal candidate to study NB as it mimics these changes. The Inventors have further recognized that the murine SCI model may have the added benefit of wide transgenic manipulations and therefore evaluation of specific pathways, as well as being cost-effective compared to larger animals.

It should be appreciated that although the evaluation experiments detailed herein employ the murine SCI model, EIM testing systems described herein may be applied to any other model and biological systems, including humans, as the present disclosure is not so limited.

In some exemplary, non-limiting embodiments, EIM may be used to detect biophysical and structural changes of the bladder detrusor muscle in subjects with NB. In exemplary experiments to evaluate the efficacy of EIM as an NB detector, female C57BL/6J mice (age 8-10 weeks, weight between 18-22 g each) may be employed, some of which may have an induced spinal cord injury to model NB.

Spinal cord injury may be induced into a subset of the mice using the following protocol. Female mice may be anesthetized using 2-3% isoflurane, and a 1 cm dorsal incision may be made to expose the thoracic spinal column. Through a mid-thoracic (T8-T10) laminectomy, the spinal cord may be transected. The incision may be closed with absorbable suture, followed by post-operative care including analgesia with buprenorphine sustained release, and 0.9% normal saline with enrofloxacin injections for three days to prevent urinary tract infections (UTIs). The bladders may subsequently be manually expressed every 12 hours for 10-14 days post-operatively, until the resumption of spontaneous voiding.

In exemplary experiments, the group of mice may be divided into two groups, the first having 10 SCI mice and 3 wild-type control mice, sacrificed at 4 weeks post SCI, and the second group having 9 SCI mice and 4 wild-type control mice, sacrificed at 6 weeks post SCI. The mice may be housed with five mice per cage, with free access to water and pelleted diet ad libitum.

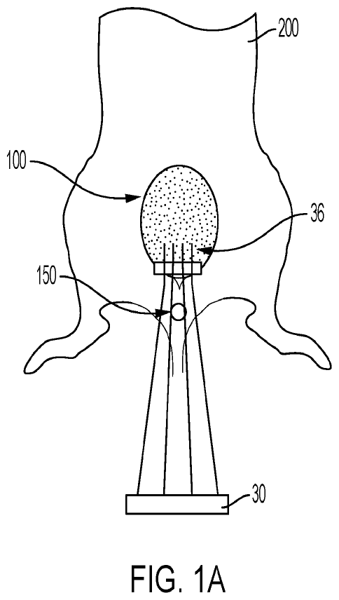

Depending on the group, four or six weeks after SCI, the mice may be euthanized via CO₂, and immediately undergo a low midline laparotomy to expose the bladder, which may then be manually decompressed to evacuate all residual urine. The bladders may subsequently be subject to EIM measurements. As shown in FIGS. 1A-1B, post-mortem EIM measurements of the decompressed bladder 100 may be performed using an impedance probe 30. The probe 30 may be inserted into a subject 200 (e.g., a mouse) through its urethral meatus 150, and subsequently positioned on the abluminal/serosal surface to puncture the detrusor muscle of the bladder 100. FIG. 1B shows a microneedle array 36 of the probe 30 gently positioned into the detrusor muscle 110. It should be appreciated that the probe width may be small enough to pass through the urethral meatus 150 without significant discomfort to the subject. In murine subjects, the probe width may be approximately 5 mm. Embodiments employing probe widths greater than or less than 5 mm are also contemplated. In some embodiments, the microneedle array 36 may be punctured approximately 1.4 mm deep into the detrusor muscle 110, but other puncture depths, above and below 1.4 mm, are also contemplated. It should be appreciated that the microneedle puncture depths of the probes described herein may depend on the test subject physiology and genetics.

As shown in FIG. 1B, in some embodiments, the microneedle array 36 may puncture the detrusor muscle 110 in a substantially normal fashion, such that the needles puncture through the muscle at an angle that is substantially normal to the muscle tissue. In other embodiments, the probe 30 and/or microneedle array 36 may be arranged to puncture the muscle 110 (or any other physiological site) at an angle, which may enable the microneedles to puncture hard-to-reach tissue, and/or reduce discomfort at the puncture site. It should be appreciated that the microneedles may puncture the detrusor muscle at any suitable angle, as the present disclosure is not limited by the angle between the target tissue and the microneedles. In some embodiments, the microneedles may be configured to only contact the tissue rather than puncture the tissue as described above. For example, the microneedles may include blunt tips that are only configured to abut the tissue such that impedance properties of the tissue surface may be measured. In such an embodiment, the microneedles may be considered to be an electrical contact pad.

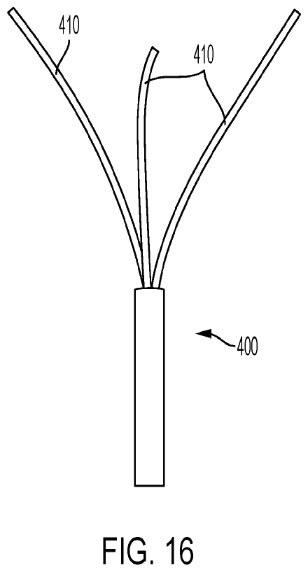

In some embodiments, a probe according to embodiments disclosed herein may include a microneedle array that is moveable between a contracted configuration and an expanded configuration. That is, the microneedle array may spread out over a larger surface of a target tissue site when in the expanded configuration. In some embodiments, the probe may be arranged such that the microneedle array is in the contracted configuration while being delivered to the tissue site and is then moved to the expanded configuration upon reaching the tissue site. The inventors have appreciated that such configurations may provide the benefit of allowing for impedance properties to be measured over a larger area of the tissue (e.g., detrusor muscle). The microneedles may be actuated between a contracted configuration and an expanded configuration in any suitable fashion as the disclosure is not so limited. For example, the microneedle array may be at least partially flexible such that the microneedles may be urged outward into the expanded configuration upon contacting the tissue surface. Such an arrangement is depicted in the exemplary embodiment of FIG. 16 which shows an end of a probe 400 having a plurality of microneedles 410. The microneedles 410 may be configured to bias outwardly upon contacting a tissue surface.

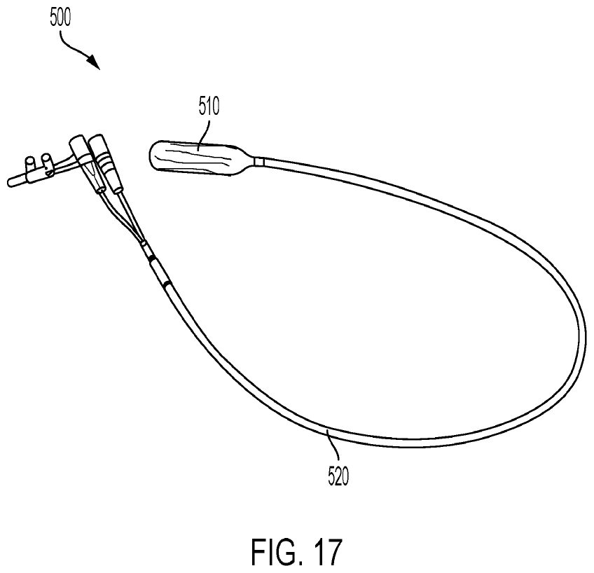

In another example, an EIM probe may include a patch disposed at an end of the probe which may include a plurality of electrical leads. The inventors have recognized that use of a patch may be beneficial to allow for impedance properties to be measured over a larger area of the tissue. Such an arrangement is depicted in FIG. 17 which shows an EIM probe 500 having EIM leads 510 on a patch disposed at the end of the probe. The EIM leads and patch may be connected to a catheter 520 to permit positioning of the probe at a bladder of the subject. In some embodiments, the patch and electrical leads may include a coating which acts as an insulating layer to limit inadvertent exposure of the electrical leads to fluids in the subject's bladder.

In addition or alternatively, the probe may include a suitable actuation mechanism, such as a spring-loaded mechanism, which is configured to bias the microneedle array between the contracted and expanded configurations. Accordingly, the probe may include a button, switch, trigger, or the like which may be operated by a user to actuate the actuation mechanism and thus bias the microneedle array. In some embodiments, the microneedles may be at least partially formed of or attached to a nitinol material to allow for such expansion.

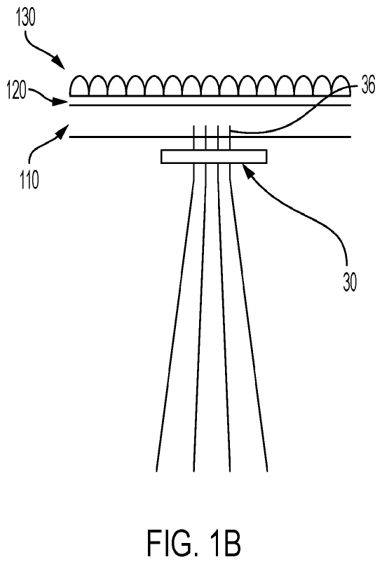

FIGS. 2A, 3A, 4A, and 5A show four exemplary embodiments of probes which may be used to non-invasively conduct real-time EIM measurements in a subject, with FIGS. 2B, 3B, 4B, and 5B showing front views of the probes. The EIM probes 3000, 3100, 3200, 3300, may each include a needle array 360 for positioning into the detrusor muscle. The needle array 360 may include four needles, as shown in FIGS. 2A-5B, but may alternatively include any suitable number of needles, greater than or less than four needles. The needles may extend substantially along the length of the probes 3000, 3100, 3200, 3300, such that electrical measurements at the detrusor muscle may be readily relayed to a processor for real-time analysis. In some embodiments, the total probe length L1 shown in FIGS. 2A-5B may be approximately 300 mm. It should be appreciated that the total probe length L1 may be less than or greater than 300 mm to accommodate the test subject. For example, the total probe length may be longer for test subjects larger than a mouse (e.g., humans). It should be appreciated that the total probe length L1 may be any suitable length, as the present disclosure is not so limited.

In some embodiments, the probe needles (which may be referred to as microneedles in some embodiments) 360 may be formed of a Tungsten material, although other conductive materials, including, but not limited to carbon fiber materials, are also contemplated. It should be appreciated that any suitable conductive material or combinations of materials, or combinations of conductive and insulating materials may be employed to form any component of the probe needles described herein. In embodiments where the probe needles are used with living subjects, the probe needles may be formed of a material compatible with the biological interface, with a low risk of cytotoxicity. In some embodiments, each probe needle may be coated with an insulating layer for electrical insulation and/or to limit the exposure of the tissue (e.g., the detrusor muscle) to the probe material. Non-limiting examples of the insulating layer include high temperature epoxies. The probe needles 360 may have, in some embodiments, a shank diameter of approximately 200 μm, which may reduce the risk of damage at the puncture site. In some embodiments, the size of the probe needles may limit any discomfort experienced by the test subject at the puncture site. It should be appreciated that the probe needles may have any suitable shank diameter, larger or smaller than 200 μm, as the present disclosure is not so limited. In some embodiments, the probe needles may be hollow, whereas in others, the probe needles may be solid. It should be appreciated that any suite form factor of the probe needles may be employed. For example, the probe needles may be 23 gauge hollow needles, but other sized needles, above and below 23 gauge, are also contemplated.

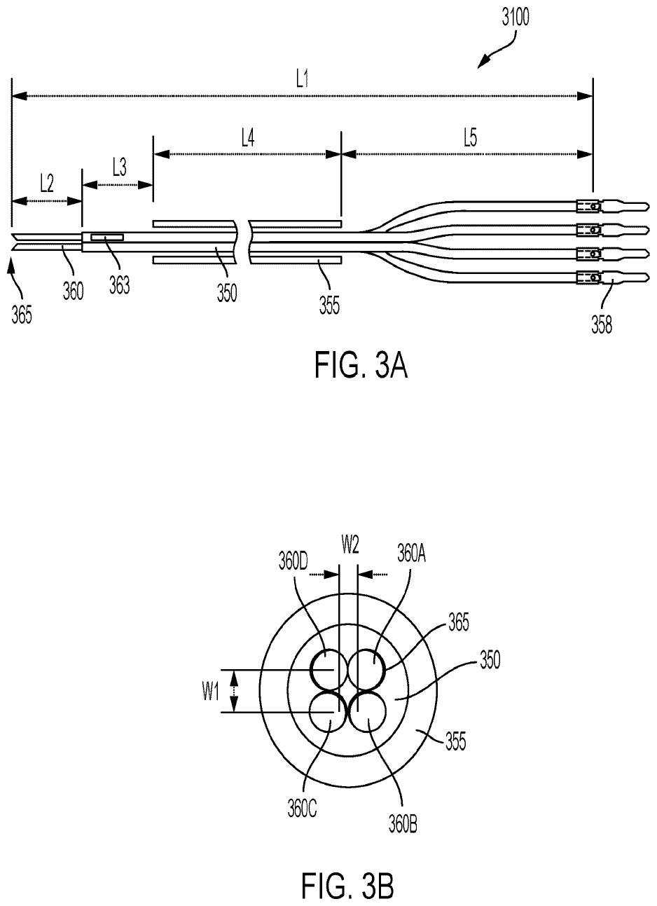

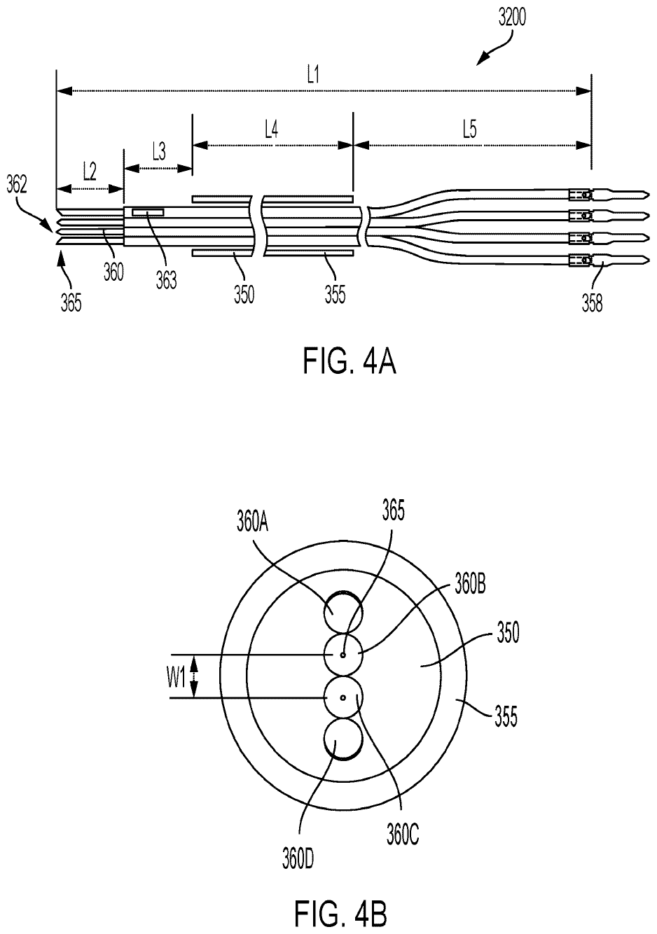

FIGS. 2B, 3B, 4B, and 5B show front views of exemplary, non-limiting embodiments of probes 3000, 3100, 3200, and 3300 respectively. The needles 360 may be arranged in any suitable layout, including in a matrix fashion, as shown in FIGS. 2A-2B, in which a first column of probe needles 360A, 360B may be arranged parallel to a second column of probe needles 360C, 360D. In some embodiments, the needles 360 may be arranged in a linear layout, as shown in FIGS. 4A-5B, in which a single column of probe needles 360A-360D may be arranged in a single column. In some embodiments, the matrix layout may result in probes with a smaller overall footprint, which may be useful for smaller subjects. Accordingly, the arrangement of the probe needles, as well as the overall geometry of the probe may be designed to accommodate a test subject. The present disclosure is therefore not limited by the arrangement of the probe needles relative to one another.

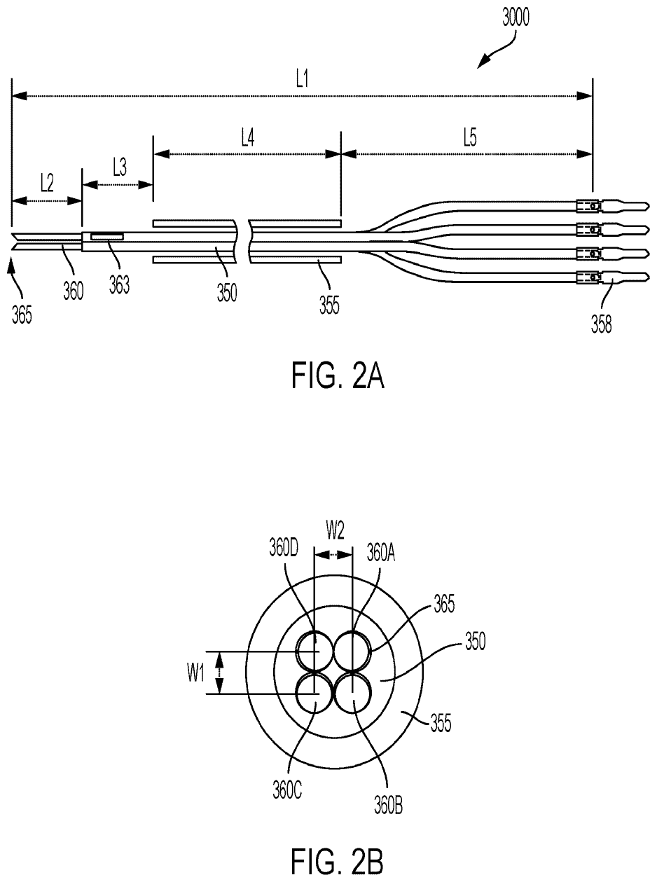

As described previously, the probe needles may puncture the detrusor muscle to collect various impedance measurements. Accordingly, the probe needles may have a tip geometry to comfortably puncture the tissue. In some embodiments, the probe needles may have beveled tips 365, as shown in FIGS. 2A-3B as well as FIGS. 5A-5B. The beveled tip may be formed using a grinding process, resulting in a bevel angle of approximately 45°. It should be appreciated that the beveled tips may be angled at any other suitable bevel angle, above or below 45°, to facilitate puncturing of the tissue, as the present disclosure is not limited by the bevel angle. In some embodiments, the probe needles may have pencil points 362, as shown in FIGS. 4A-4B. The pencil points 362 may have an axially symmetric shape akin to a point of a pencil, for easy puncturing, as shown in FIGS. 4A-4B, but asymmetric pencil points are also contemplated. FIGS. 4A-4B show a probe 3200 with a combination of two bevel tipped 365 probe needles and two pencil point 362 probe needles. The probes of the present disclosure may have any suitable combination of probes having any suitable probe tip shapes (non-limiting examples include beveled tip and pencil point shapes), as the present disclosure is not so limited.

In some embodiments, the probe needles may be multifunctional, such that in addition to measuring impedance properties of the target tissue, the probe needles may also be employed to measure other tissue properties such as temperature, pressure, and/or electric activity (e.g., through an electromyography test). The probe needles may be used to measure any suitable property of the tissue in addition to, or separate from, tissue impedance. In some embodiments, one or more secondary needles may be used to measure tissue properties, while the primary probe needles measure tissue impedance.

As shown in the front views of FIGS. 2A, 3A, 4B, and 5B, the probe needles 360A-D may have a spacing width W1 measured center to center between the probe needles. In some embodiments, the spacing width W1 of the probe needles may be 305 μm, although other spacing widths, greater than or less than 305 μm are also contemplated. The spacing width W1 of the probe needles may account for the shank diameter of each probe needle, as discussed earlier, as well as an insulating tubing 350, as shown in FIGS. 2A-5B. The insulating tubing 350, which may be coated over or replace the epoxy insulating layer noted previously, may be formed of an insulating polymer material such as polyimide. In some embodiments, at least one of the probe needles 360 may include a label 363 arranged on top of the insulating tubing 350, to distinguish one of the probe needles and orient the operator. In some embodiments, the label 363 may indicate a first probe needle 360A, but other label arrangements and combinations of arrangements are also contemplated.

In some embodiments, one or more of the probe needles 360A-D may be input needles, serving to deliver an electrical signal to the target tissue, whereas others may serve to readout the impedance signal. For example, in FIG. 2B, needles 360A and 360D may be input needles while needles 360B and 360C may be readout needles. It should be appreciated that any combination of pairs of needles may be employed for either signal delivery and/or readout, as the present disclosure is not so limited.

The Inventors have appreciated that maximizing the distance between input needles and readout needles may enhance the signal quality of the probe. Thus, in some embodiments, as represented by FIG. 3B, the needle tip 365 of needles 360B and 360C may be rotated, which may reduce a width W2 in between needles 360B and 360C in comparison to embodiments represented by FIG. 2B. It should be appreciated that the needle tips may be arranged in any configuration relative to each needle's longitudinal axis, which may enhance the readout signal and/or reduce noise.

In some embodiments, such as those shown in FIGS. 4B and 5B, the distance between signal needles 360A and 360D and readout needles 360B and 360C can be further maximized by arranging the needles in a linear fashion, in order to enhance the readout signal and/or reduce noise, compared to the more compact configuration of FIGS. 2B and 3B. Therefore, depending on the application (e.g., target site, patient size, etc.), the size and signal quality may be optimized by re-arranging the probe needles accordingly. As such, the probes of the present disclosure are not limited by the number, arrangement, or size of the probe needles.

In some embodiments, the probe needles 360 may extend beyond the insulating tubing 350 by a length L2, as shown in FIGS. 2A, 3A, 4A, and 5A. This length L2 may be adjusted to achieve a desired puncture depth for a test subject. As noted previously, an exemplary puncture depth is 1.4 mm, although puncture depths greater than or less than 1.4 mm are also contemplated. Accordingly, the length L2 may be greater than the puncture depth by a safety factor or other buffer to control the puncture depth. In some embodiments, the length L2 may be 10 mm, although embodiments greater than or less than 10 mm are also contemplated.

As disclosed herein, the EIM probes may include EIM sensors and electrical leads that are coupled with an array of microneedles, where the array of microneedles may contact or puncture a subject's detrusor tissue to collect impedance measurements. The inventors have recognized that, in some cases, the leads and/or portions of the microneedles may be inadvertently exposed to fluids in a subject's bladder during operation of the EIM probes. Thus, the inventors have appreciated benefits associated with providing a coating on the leads and/or microneedles to provide an insulating layer to limit the exposure of the leads and/or microneedles to the detrusor tissue and bladder fluids. In some embodiments, the coating may be arranged such that only the tips of the microneedles are exposed to contact the detrusor tissue. This may be accomplished by extending the insulating tubing 350 into the region defined by length L2 such that the tubing 350 coats a larger amount of the probe needles 360. While such an example is disclosed, the amount of insulating tubing 350 may be increased or decreased as needed to coat a larger or smaller region of the probe needles 360, respectively, as the disclosure is not so limited.

In some embodiments, the probe needles 360, coated with the insulating tubing 350, may be further encapsulated by an outer tubing layer 355. In some embodiments, the outer tubing layer 355 may be formed of a stainless steel material, although other electrically conductive materials are also contemplated. In some embodiments represented by FIGS. 2A-3B, the outer tubing layer 355 may be a 19 gauge thin wall (19TW) tube, with a nominal outer diameter of approximately 1.1 mm and an inner diameter of approximately 0.8 mm.

In some embodiments represented by FIGS. 4A-5B, the outer tubing layer 355 may be a 17 gauge extra thin wall (17XTW) tube, with a nominal outer diameter of approximately 1.5 mm and a nominal inner diameter of approximately 1.3 mm. It should be appreciated that the larger gauge tube of FIGS. 4A-5B may accommodate the linear layout of the probe needles, whereas the smaller gauge tube of FIGS. 2A-2B may minimize the probe diameter and subsequently, the overall footprint of the probe, which may be suitable for smaller test subjects.

In some embodiments, the probe may be introduced into the test subject (e.g., through their urethral meatus) using endoscopic instruments (e.g., cystoscopes). Accordingly, the outer tubing layer may be designed to be inserted into a sheath or hollow tube of the endoscopic instrument. Alternative methods of introducing the probe into the test subject are also contemplated, as the present disclosure is not so limited.

The outer tubing layer 355 may extend along the longitudinal axis of the probe a distance L4, as shown in FIG. 2A-5B. In some embodiments, the outer tubing layer length L4 may be any suitable length, including, but not limited to, approximately 250 mm. As discussed earlier, the various lengths of the probe may be adjusted in any suitable direction to accommodate a test subject. Accordingly, outer tubing layer lengths greater than or less than 250 mm are also contemplated.

Similarly, the protrusion length of the insulating tubing 350 from the outer tubing layer 355, shown as length L3 in FIGS. 2A-5B, may be any suitable length to accommodate a test subject. In some embodiments, the length L3 may be approximately 10 mm, but lengths greater than or less than 10 mm are also contemplated. At an opposing end of the probe, as shown in FIGS. 2A-5B, the insulating tubing 350 may extend beyond the outer tubing layer 355, towards a set of pins 358, for a length L5. The length L5 may be any suitable length, including, but not limited to 30 mm, although lengths less than or greater than 30 mm are also contemplated.

It should be appreciated that any of the geometric parameters described herein may be adjusted to accommodate any suitable test subject or any other measurement parameter, as the present disclosure is not limited by the geometric parameters of the probes. For example, the probe design may be adjusted to account for phenotypic and genetic variability and design with reasonable cost. In another example, the probe's width may be adjusted to accommodate a test subject's urethral meatus (see FIGS. 1A-1B) without significant discomfort. The probe width may be any suitable size, greater than, equal to, or less than approximately 5 mm. As described previously, the probe may be passed into the test subject using existing endoscopic instruments, including, but not limited to, cystoscopes.

In some embodiments, the probes may include pins 358 positioned at their distal end, which may be electrically connected to the probe needles 360 positioned at an opposing end (e.g., proximal end) of the probes. In some embodiments, the pins 358 may transport electrical signal from the probe to a processor for real-time analysis of the detrusor muscle impedance behavior.

As described previously, the probes of the present disclosure may measure any suitable impedance variables, including, but not limited to resistance, reactance, and phase. Without wishing to be bound by theory, impedance resistance may include the measurement of difficulty passing current through the tissue, impedance reactance may include the measurement of the capacitive effects of the cell membranes, and impedance phase may include a trigonometric proportion of the resistance and reactance, such that phase may be equivalent to the arctangent of the quotient of the reactance and the resistance.

EIM readings may be conducted using any suitable probe described herein. In some embodiments, the EIM measurements may be collected between 1 kHz and 1 MHz given the occurrence of artifacts at the extremes of the frequency range. However, measurements taken at other frequency ranges are also contemplated.

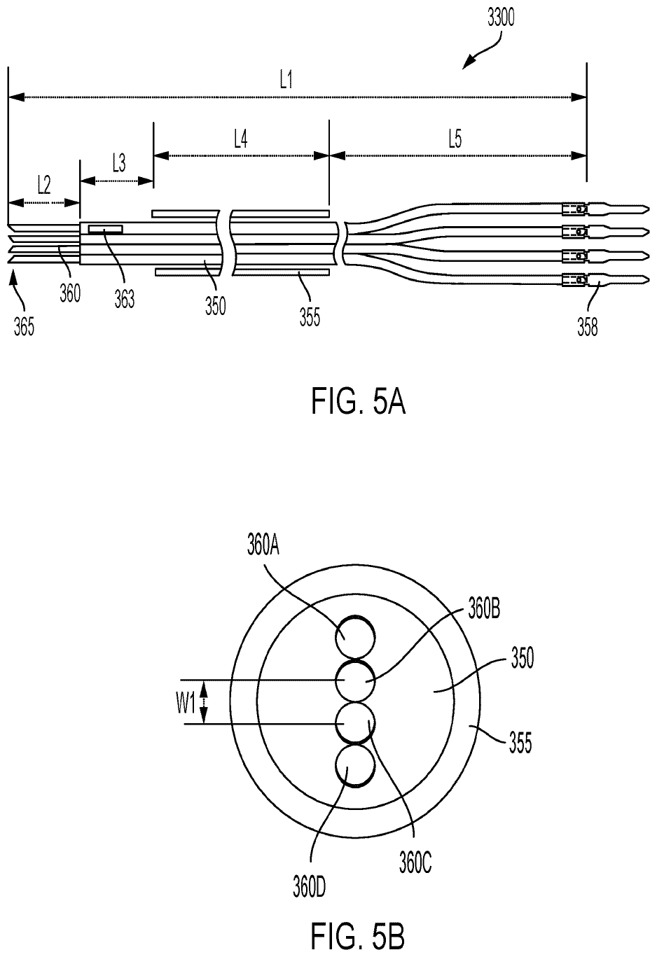

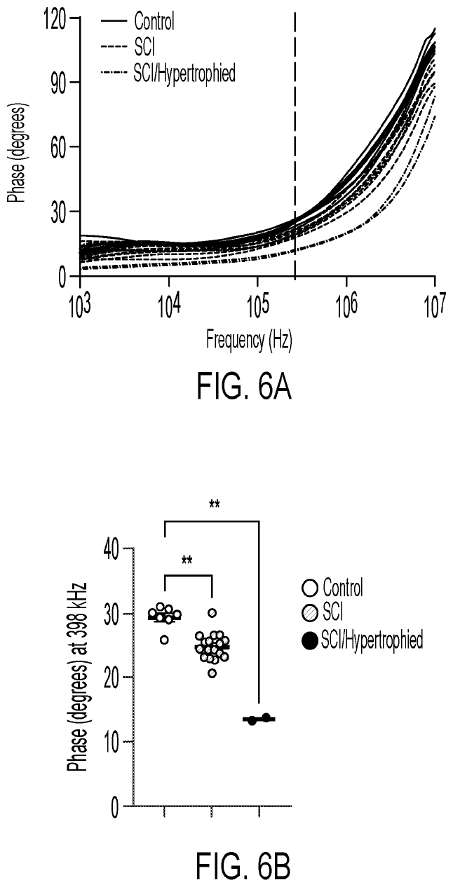

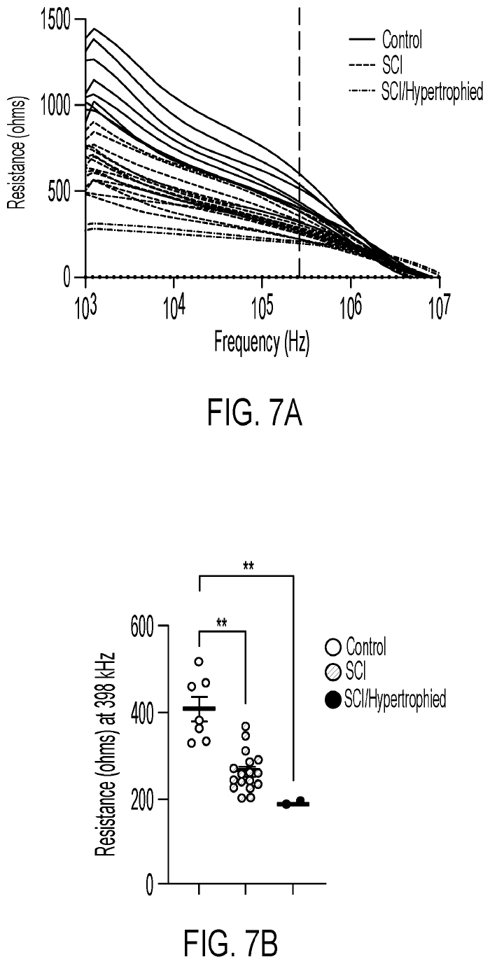

FIGS. 6A-6B represent exemplary phase measurements of bladders in the control group and the two SCI groups (including the 4-week and 6-week groups described earlier), including the hypertrophied samples from the 6-week group. As described earlier, the hypertrophied samples exhibited severely thickened bladder walls and bladder stones, with significantly higher bladder to weight ratios compared to other SCI bladders. FIGS. 7A-7B represent exemplary resistance measurements from the same group, and FIGS. 8A-8B represent exemplary reactance measurements from the same group. FIGS. 6B, 7B, and 8B represent values at 398 kHz frequency from FIGS. 6A, 7A, and 8A, respectively, for visual clarity.

As shown in the exemplary and non-limiting measurements of FIGS. 6A-8B, compared to the control group, the SCI groups are associated with significantly lower phase, lower resistance, and lower reactance measurements. For phase measurements, shown in FIGS. 6A-6B, the clearest separation between SCI and control group may be observed between 10⁵-10⁶ Hz. For resistance, shown in FIGS. 7A-7B, and reactance, shown in FIGS. 8A-8B, the control group values may be higher than the SCI values across most of the frequency spectrum. As shown, the two SCI mice from the 6-week group demonstrating hypertrophy exhibited lower phase, resistance, and reactance than the SCI mice across the entire measured spectrums of FIGS. 6A-8B.

It should be appreciated that the measurements shown in FIGS. 6A-8B may be collected with any of the probes described herein, including, but not limited to, probes 3000, 3100, 3200, and 3300 of FIGS. 2A-5B.

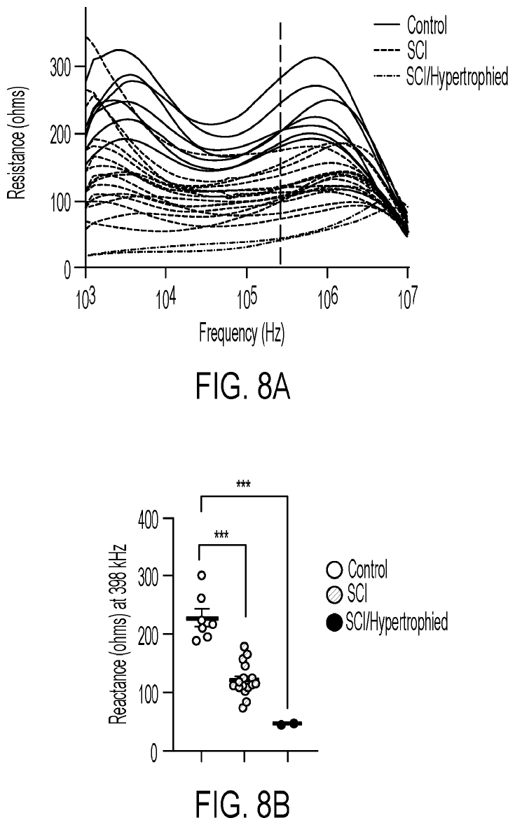

FIG. 9 shows an exemplary simplified graphic depicting the differences in phase behavior between the three bladder types, control, SCI group, and a group including the two mice from the 6-week group which exhibited severe hypertrophy. FIG. 9 shows, in a more visually clear manner, the distinct differences in behavior between the three groups across a smaller frequency spectrum. As shown in FIG. 9, the control group may exhibit the highest phase measurements whereas the SCI group may exhibit a reduced phase measurement in comparison, with the severe hypertrophy SCI group exhibiting the lowest phase measurements.

The exemplary data shown in FIGS. 6A-9 suggests that EIM probes may be used to detect NB conditions through a variety of EIM measurements. To ensure that the detected levels of NB with EIM, which may be conducted non-invasively, accurately represent the function of the bladders, a series of conventional verification experiments, both qualitative and quantitative, may be employed to compare with the EIM measurements.

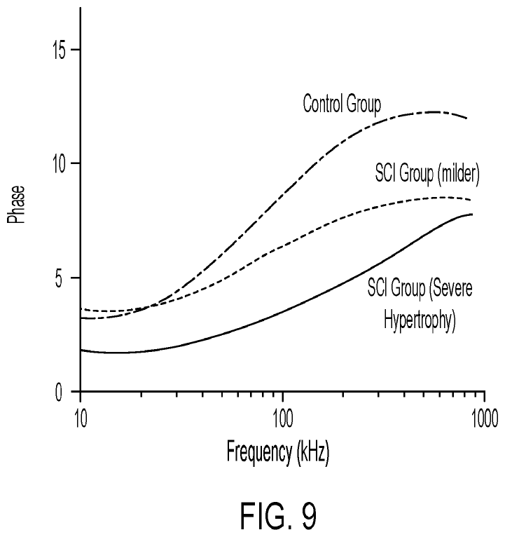

Following the EIM reading, the bladders may be harvested and weighed to evaluate basic physiological measurements. In some embodiments, as shown in FIG. 10, at 4 weeks, the SCI mice may have a significantly higher bladder weight while the body weight may be similar to the control mice. As shown in FIG. 10, the mean bladder-to-body weight ratio may be approximately 0.0036 in SCI mice bladders compared to 0.0007 in control mice bladders, with p<0.001, as indicated by four asterisks. In some embodiments, at 6 weeks, the mean body weight may be higher. For example, the mean body weight may be 18.9 g for the 6-week group compared to 16.7 g for the 4-week group. Similar to the 4-week group, the 6-week group may have a mean bladder-to-body weight ratio of 0.0038 for the SCI mice bladders compared to 0.0007 for the control mice bladders, with p<0.001. In some embodiments, 2 of the 9 SCI mice of the 6-week group may develop special bladder hypertrophy phenotype with severely thickened bladder wall and bladder stone inside of the bladder, which may lead to a bladder to body weight ratio almost twice as large compared to the other SCI mice, at 0.006. According to the data presented in FIG. 10 and described herein, the SCI mice may exhibit greater physiological markers (e.g., enlarged bladders) for NB compared to the control group, with the hypertrophied SCI bladders exhibiting even more significant markers. Accordingly, the physiological measurements of bladder weight correspond with the EIM measurements shown in FIGS. 6A-9 in terms of trends, validating, at least partially, the use of EIM measurements for evaluation of NB.

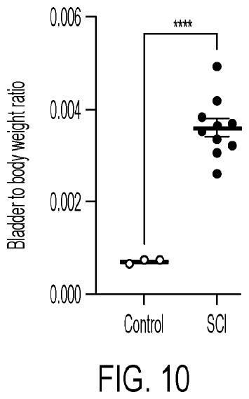

In some embodiments, the functional state of the bladders may be qualitatively evaluated prior to euthanization. For example, the functional state of the bladders may be evaluated using a voiding spot assay to better understand how much control the mice may have over their bladders. In some embodiments, at 4 weeks after SCI, individual mice may be placed in a rectangular polycarbonate cage without wire flooring, lined with Whatman paper on for several hours a day (e.g., from 8 μm to 12 am). The mice may be fed ad libitum, but water may be restricted during this time to avoid water dripping onto the filter paper. The lining papers may subsequently be dried and imaged using UV illumination to visualize voided spots of urine. FIG. 11 is an exemplary voiding spot assay contrasting the constant dribbling of urine across the cage for the SCI group compared to the control group, which may demonstrate fewer voiding spots. In some embodiments, the lower number of voiding spots may indicate more volitional and controlled voiding. Qualitatively, the voiding assay may provide information regarding the functionality of the murine bladders prior to euthanization. The lack of control qualitatively shown in FIG. 11 further matches the changes in EIM measurements outlined in FIGS. 6A-9, validating, at least partially, the use of EIM measurements for evaluation of NB.

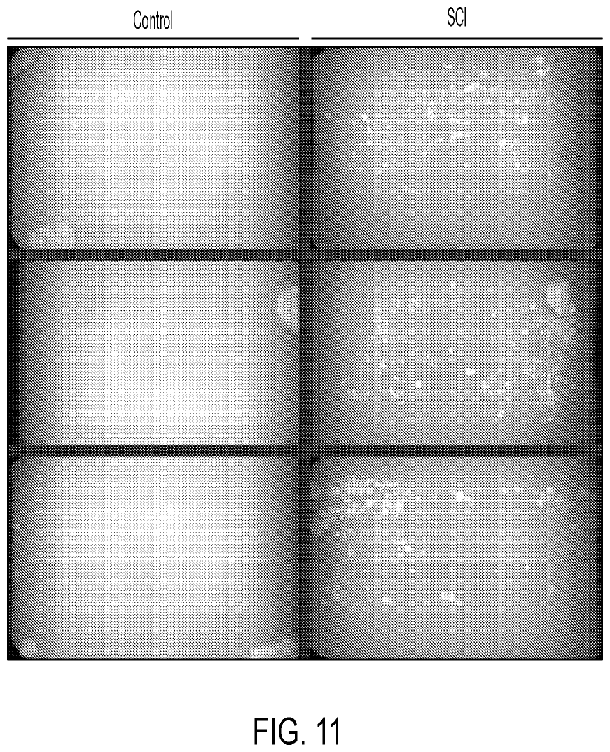

Another qualitative evaluation of the bladders may include tissue histology. In some embodiments, the harvested bladders may be equilibrated in a buffered saline solution and then stabilized using flash freezing in liquid nitrogen or formalin fixation. The tissue may then be embedded in paraffin and sectioned into 8 micron thick slices for staining (e.g., Masson's Trichrome staining) and imaging. FIGS. 12A-12B show exemplary histological samples between a control mouse bladder (FIG. 12A) and a SCI mouse bladder (FIG. 12B), with annotations of areas including the detrusor muscle (D), the lamina propria (LP), the urothelium (U), and lumen (L). For reference, FIG. 1B shows the needle array 36 relative to the detrusor muscle 110, the lamina propria 120 and the urothelium 130. FIG. 12A shows the detrusor being thinner and more homogenous with the muscle (stained in red), compared to the SCI model represented by FIG. 12B. Once again, the qualitative differences in the tissue histology of control and SCI bladders may further validate, at least partially, the use of EIM measurements for evaluation of NB.

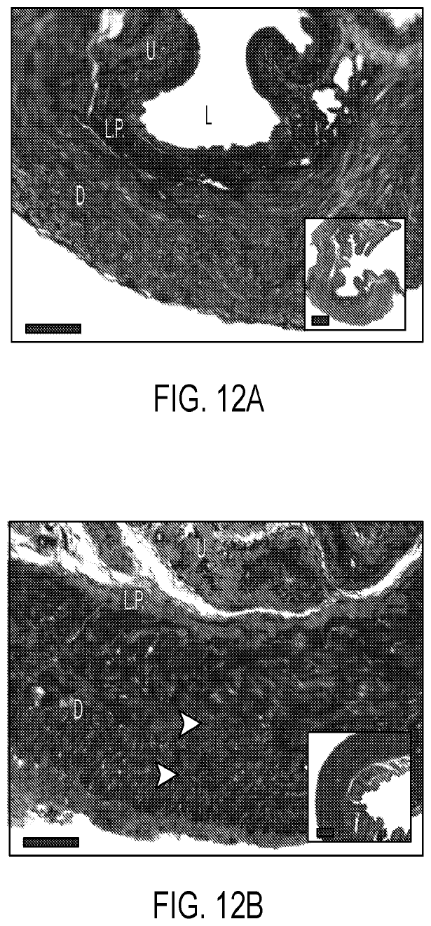

In some embodiments, quantitative information may be derived from the histological studies of the bladder tissue. For example, an image processing macro may be developed to identify the concentration of the fiber-stain indicative of collagen from the histological images of FIGS. 12A-12B. An integrated density of collagen signal in the detrusor, which may be normalized to the detrusor area as a fraction of the total tissue area, is shown in FIG. 13 for both control and SCI mouse bladders. The data presented in FIG. 13 suggests that the SCI bladder detrusor layer may be significantly thickened with collagen deposition (blue infiltration, as shown in FIG. 12A) compared to the uniformed detrusor smooth muscle layer (homogeneous red, as shown in FIG. 12B) in the control group. Compared to control, SCI may accordingly be associated with a higher density of collagen in the detrusor layer. For example, as shown in FIG. 13, the SCI model may have 1.9 times greater collagen in the detrusor layer compared to the control bladders, with a p=0.009.

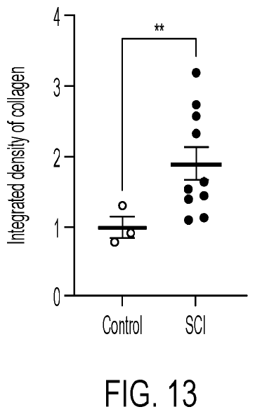

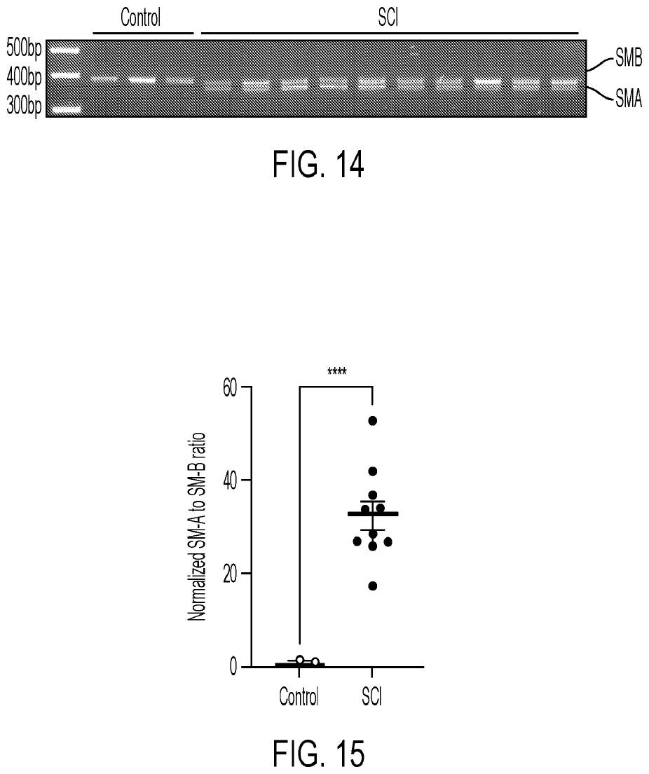

In some embodiments, the thickening of the detrusor layer shown in FIGS. 12A-12B may be further evaluated using mRNA expression. In exemplary experiments, bladder tissue may be homogenized and further treated for RNA isolation per conventional methods known in the art. The concentration of RNA in a sample may be quantified using a small volume spectrophotometer (and/or any other suitable technique) to ensure a minimum concentration of RNA for reverse transcription into cDNA using conventional techniques known in the art. To quantitatively evaluate the differences between the control and SCI model bladders, the levels of smooth muscle myosin heavy chain (SMMHC) isoforms SMA and SMB may be assayed. In some embodiments, a semi-quantitative reverse transcription polymerase chain reaction (RT-PCR) using gene-specific primers (SM-A/SM-B, forward (5′-CCA CAA GGG CAA GAA AGA CAG C-3′) and reverse (5′-TCC GGC GAG CAG GTA GAA GA-3′)) may be run on agarose gel. FIG. 10 shows an exemplary RT-PCR gel for control and SCI model bladders, as indicated. The relative expression levels of the gel in FIG. 14 may be quantified using imaging processing software, resulting in the data presented in FIG. 15. As shown in FIG. 14, the control group bladder only expressed SM-B, whereas SCI group bladders expressed both SM-A and SM-B, resulting in a higher SM-A to SM-B ratio (32.5 in SCI vs 1 as control, p<0.001), as shown in FIG. 15.

The quantitative distinction of collagen density between the control and SCI samples, described relative to FIGS. 12A-13, may therefore further validate the EIM measurements outlined in FIGS. 6A-9.

The exemplary data presented in FIGS. 10-15 may validate the exemplary EIM signatures shown in FIGS. 6A-9, suggesting that EIM measurements may have the potential to identify bladder detrusor changes from SCI, with distinct differences in all 3 major EIM parameters being observed. Compared to normal controls, SCI mice bladder may have distinct EIM signature with lower resistance, lower reactance, and lower phase values, as shown in FIGS. 6A-8B. It should be appreciated that the measurements identified behavior that may be different from typical increased resistance in muscle atrophy with reduced muscle/circuitry, as indicated by the neurogenic bladder changes such as collagen tissue deposition, tissue edema, inflammation in detrusor, all of which may be responsible for the reduced resistance measurement in SCI group (see FIGS. 7A-7B) in addition to reduced reactance and phase. Such structural variations may be identified by increased collagen deposition, which may be verified by the exemplary experiments shown in FIGS. 12A-13, and specific mRNA expressions, which may be verified by the exemplary experiments shown in FIGS. 14-15, indicating bladder remodeling in the neurogenic bladders of the SCI group.

In some embodiments, the hypertrophic subtype of the SCI bladders may exhibit more prominent differences compared to control animals and to milder phenotypes. The EIM measurements depicted in FIG. 9 suggest that the control group may exhibit a higher phase compared to the SCI injured animals, with the severe hypertrophy SCI group having the lowest phase measurement. It should be appreciated that while phase may be uniformly lower in all skeletal muscle diseased states, as shown in FIG. 9, resistance and reactance values, which constitute the phase measurements, may be less predictable across the diseases. In some embodiments, resistance may be reduced due to the conductive property of collagen, which may also reduce the reactance values due to a reduction in the “chargeability” of the tissue, given the presence of cells being replaced by collagen.

In some embodiments, the validation data of FIGS. 10-14B, which quantitatively and qualitatively evaluate the state of NB according to conventional methods, may align with the EIM data presented in FIGS. 6A-10. Accordingly, this comparison suggests that EIM may serve as a biomarker in SCI and neurogenic bladder research, providing precision and real-time results compared to the limited conventional tools for phenotyping NB. As described earlier, EIM methods may provide a real-time, reliable, repeatable, objective, and non-invasive option for NB patients.

It should be appreciated that although a murine model was described relative to the experiments outlined in FIGS. 6A-15, any other suitable model, including porcine and human models, may also be employed. It should also be appreciated that NB may present itself in other models (e.g., in humans) in a variety of different types of cases, which may not all be represented by the murine SCI model.

As described earlier, in some embodiments, the probes and EIM measurements described herein may enable a treating clinician to evaluate and phenotype bladder detrusor tissue in a real-time, non-invasive manner to facilitate and streamline diagnosis. In some embodiments, the probes and EIM measurements may also be used in conjunction with bladder treatments, which conventionally involve Botox or biomaterial injection, enabling the treating clinician to identify optimal injection sites. In some embodiments, EIM data may be used to develop predictive algorithms to help predict treatment response and further enhance the capability to improve treatment efficacy with minimal injection volumes to expedite treatment, reduce costs, and improve the overall safety of the treatment.

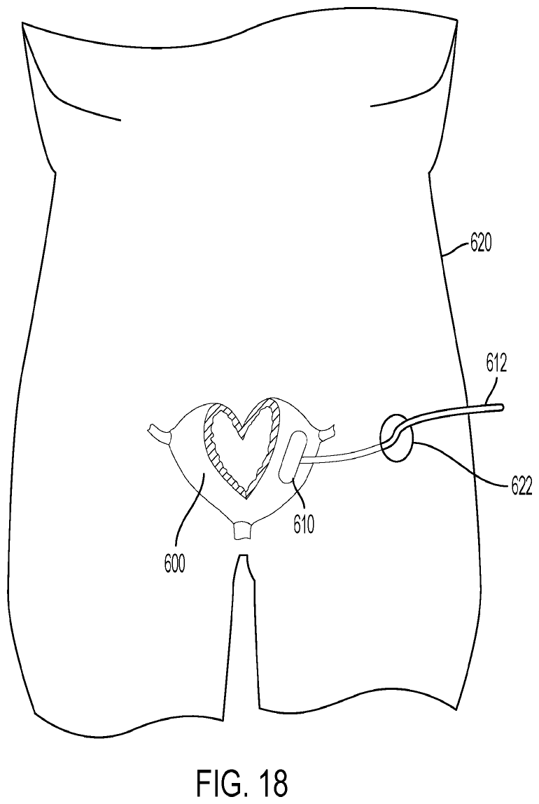

In addition, while the embodiments disclosed herein are primarily described in reference to collecting measurements from the bladder detrusor tissue by puncturing the tissue, the inventors have also appreciated that intraoperative measurements can be collected from the outer layer (i.e., the outside) of the bladder. That is, EIM probes may be placed on the outer layer of the bladder to analyze the tissue characteristics of the outer layer, which may help to inform operative decisions of clinicians. For example, the tissue characteristics of the outer layer may be useful for clinicians in cases of bladder exstrophy. FIG. 18 shows an embodiment where an EIM probe 610 is positioned on an outer layer of the bladder 600. The EIM probe 610 may be connected to a catheter 612 which is inserted through a hole 622 in the abdomen of a subject 620. While such an arrangement is disclosed, the EIM probe may be inserted into the subject to contact the subject's bladder in any suitable fashion (e.g., through the subject's urethra) as the disclosure is not so limited. In addition, in some embodiments, the EIM probes may be placed on an inner layer (i.e., the inside) of the bladder as the disclosure is not so limited.

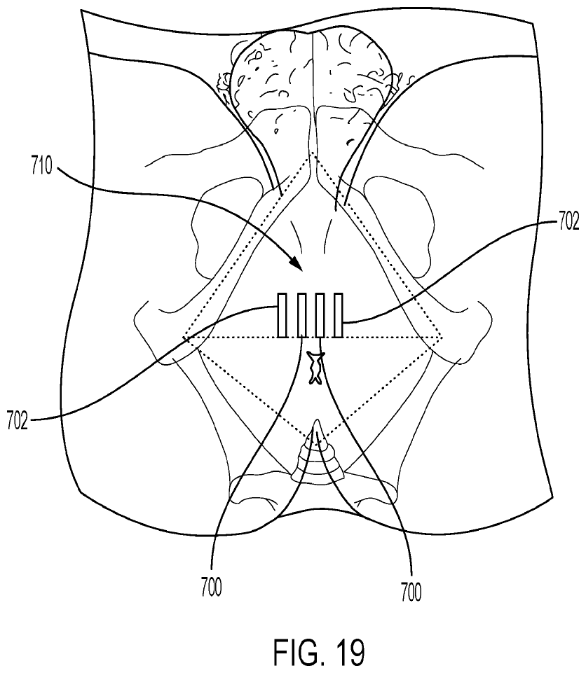

In addition or alternatively to measurements collected from a subject's bladder tissue, impedance measurements may be collected from a surface of the subject's perineum. These surface measurements may be used to assess the subject's pelvic floor to provide an indirect indication of bladder function of the subject. For example, FIG. 19 shows a plurality of impedance electrodes 700 and 702 which may be positioned on a perineum 710 of a subject. In some embodiments, the outer electrodes 702 may be configured to deliver electrical current to the perineum (i.e., current emitting) while the inner electrodes 700 may be configured to collect the resultant signals (i.e., voltage sensing). The inventors have appreciated that such an arrangement may allow the perineal musculature to be analyzed to provide information on the subject relating to substantial hypertrophy, atrophy, fibrosis, and other suitable histological changes as the disclosure is not so limited. In turn, information that is collected relating to the perineal musculature may be used to monitor treatments and progression of the subject. While the above example is disclosed, the electrodes may be arranged in any suitable fashion as the disclosure is not so limited. For example, the outer electrodes may be voltage sensing while the inner electrodes may be current emitting.

In some embodiments, a plurality of impedance electrodes may be placed on a perineum of the subject while EIM probes having impedance electrodes may contact the bladder tissue of the subject (e.g., a plurality of microneedles contacting the detrusor tissue). In some embodiments, the data collected for the perineum and the bladder of the subject may be used in combination to provide a holistic analysis of the subject's bladder function. For example, during a bladder filling procedure (e.g., hydrodistension), the bladder may stretch and cause fluctuation in a subject's EIM measurements while tightness of the subject's sphincter may cause perineal EIM measurements to remain relatively unchanged. Accordingly, the inventors have recognized that the combination of perineum and bladder tissue data may be useful in diagnosing and monitoring urinary function and condition of the subject.

The inventors have also appreciated that benefits may be realized by incorporating the embodiments of EIM probes described herein into a device that is wearable by a user. Specifically, a wearable device may be employed to detect changes and collect measurements in the detrusor muscle over time while a user goes about their daily activities. Such an arrangement may be used to provide information on the bladder function of the user over time as well as information on the bladder volume of the user. In some embodiments, the wearable device may include a catheter (e.g., a pigtail catheter) that is deployed into the bladder with an EIM probe having a microneedle array. The wearable device may be affixed to a portion of the user's body (e.g., thigh, abdomen, etc.) or clothing in any suitable fashion (e.g., adhesive, fasteners, etc.). In some embodiments, the wearable device may also be configured to collect impedance measurements from a perineum of the subject. The data collected from the subject's perineum may be used in combination with the data collected from the subject's bladder to provide a holistic analysis of the subject's bladder function as described above. The wearable device may also include a housing configured to receive one or more components of the device including, but not limited to a processor for real-time analysis of the electrical measurements at the detrusor muscle. In some embodiment, the wearable internal bladder device described above may be worn in conjunction with a device that collects measurements of a subject's perineum as the disclosure is not so limited.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

The foregoing description of various embodiments are intended merely to be illustrative thereof and that other embodiments, modifications, and equivalents are within the scope of the invention recited in the claims appended hereto.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

1. A method of evaluating bladder function, the method comprising:
contacting bladder detrusor tissue of a subject with a plurality of microneedles arranged at a proximal end of a probe; and
collecting at least one electric impedance myography measurement from the bladder detrusor tissue.

2. The method of claim 1, further comprising arranging the probe at least partially within an endoscopic instrument.

3. The method of claim 1, further comprising passing the probe through a urethral meatus of the subject.

4. The method of claim 1, wherein collecting at least one electric impedance myography measurement comprises collecting at least one of an impedance reactance measurement and an impedance resistance measurement.

5. The method of claim 1, further comprising processing the at least one impedance myography measurement with at least one processor.

6. The method of claim 1, wherein contacting the bladder detrusor tissue includes puncturing the bladder detrusor tissue with the plurality of microneedles.

7. The method of claim 1, further comprising contacting an outside of the bladder with the plurality of microneedles.

8. The method of claim 1, further comprising contacting an inside of the bladder with the plurality of microneedles.

9. The method of claim 1, wherein the plurality of microneedles arranged at the proximal end of the probe are configured to move between a contracted configuration and an expanded configuration.

10. The method of claim 1, further comprising collecting at least one electric impedance myography measurement from a perineum of the subject, and further comprising processing each of the at least one impedance myography measurement from the bladder detrusor tissue and the at least one impedance myography measurement from the perineum with at least one processor.

11. (canceled)

12. A method of improving bladder function, the method comprising:
contacting bladder detrusor tissue of a subject with a plurality of microneedles arranged at a proximal end of a probe;
collecting at least one electric impedance myography measurement from the bladder detrusor tissue; and
injecting a material into the bladder detrusor tissue.

13. The method of claim 12, wherein the material is at least one of the group comprising of Botox and a biomaterial.

14-23. (canceled)

24. An apparatus for evaluating bladder function, the apparatus comprising:
a probe comprising a plurality of microneedles configured to contact bladder detrusor tissue of a subject,
wherein the probe is configured to collect at least one electric impedance myography measurement from the bladder detrusor tissue.

25. The apparatus of claim 24, wherein the at least one electric impedance myography measurement includes at least one of an impedance reactance measurement and an impedance resistance measurement.

26. The apparatus of claim 24, wherein the probe is configured to pass through a urethral meatus of the subject.

27. The apparatus of claim 26, wherein the probe is configured to pass through at least a portion of an endoscopic instrument.

28-29. (canceled)

30. The apparatus of claim 24, wherein the plurality of microneedles are configured to puncture the bladder detrusor tissue of the subject.

31-32. (canceled)

33. The apparatus of claim 24, wherein the plurality of microneedles arranged at the proximal end of the probe are configured to move between a contracted configuration and an expanded configuration.

34. The apparatus of claim 24, further comprising a plurality of impedance electrodes positioned at a perineum of the subject, wherein the plurality of impedance electrodes are configured to collect at least one electric impedance myography measurement from the perineum of the subject.

35. The apparatus of claim 34, further comprising a processor configured to process each of the at least one impedance myography measurement from the bladder detrusor tissue and the at least one impedance myography measurement from the perineum.

36. The apparatus of claim 24, further comprising a plurality of electrical leads connected to a plurality of electric impedance myography sensors, wherein at least one of the plurality of electrical leads and the plurality of microneedles are at least partially coated with an insulating material.

37. The apparatus of claim 24 in combination with a wearable device configured to be worn by the subject.
